(19) United States
(12) Patent Application Publication
(10) Pub. No.: US 2014/0057295 A1
(43) Pub. Date: Feb. 27, 2014

(54) ANTI-MULLERIAN HORMONE CHANGES IN PREGNANCY AND PREDICTION OF ADVERSE PREGNANCY OUTCOMES AND GENDER

(71) Applicants: Barbara J. Stegmann, Iowa City, IA (US); Donna Ann Santillan, Iowa City, IA (US); Mark K. Santillan, Iowa City, IA (US)

(72) Inventors: Barbara J. Stegmann, Iowa City, IA (US); Donna Ann Santillan, Iowa City, IA (US); Mark K. Santillan, Iowa City, IA (US)

(21) Appl. No.: 14/001,687

(22) PCT Filed: Feb. 28, 2012
(86) PCT No.: PCT/US2012/026913

§ 371 Date: Nov. 13, 2013

Related U.S. Application Data
(60) Provisional application No. 61/447,488, filed on Feb. 28, 2011.

(51) Int. Cl. G01N 33/74

(52) U.S. Cl. CPC G01N 33/74
USPC 435/7.92

(57) ABSTRACT

The present invention provides for methods for evaluating the risk of an adverse pregnancy outcome in a subject and methods for treating subjects evaluated as being high risk. In some aspects, the present invention provides a method of evaluating the risk of an adverse pregnancy outcome in a subject, where if the subject does have an abnormal level of AMH as compared to a predetermined normal level the subject is more likely to have an adverse pregnancy outcome, and if the subject does not have an abnormal level of AMH the subject is less likely to have an adverse pregnancy outcome. In other aspects, the present invention provides a method of determining the gender of a fetus comprising obtaining information regarding the level of AMH in a sample from a pregnant subject.

### CROSS REFERENCE TO RELATED APPLICATION

This application claims priority to U.S. Provisional Patent Application Ser. No. 61/447,488 filed Feb. 28, 2011. This provisional application is expressly incorporated by reference.

### BACKGROUND OF THE INVENTION

1. Field of the Invention

The present invention relates generally to the field of biology. More particularly, it relates to devices and methods for identifying subjects at risk for an adverse pregnancy outcome as determined by the level of Anti-Müllerian Hormone in a sample.

2. Description of the Related Art

Over 4 million women give birth annually in the United States, and over 500,000 of these babies will be born prematurely (Heron et al., 2007). The risk of preterm birth increases in women who suffer from abnormal feto-placental signaling (Silasi et al., 2010). Although the etiology of abnormal feto-placental signaling begins early in gestation (Silasi et al., 2010, Meanwell et al., 2009, Savitz 2008), the consequences are not evident until much later. Currently, there are no adequate methods available to evaluate early feto-placental development. Development of screening tests that could be used early in pregnancy to predict women at risk for preterm deliveries or other adverse pregnancy outcomes would allow for close surveillance in these women and improve obstetric outcomes (Wang et al., 2009).

### SUMMARY OF THE INVENTION

The present invention provides for methods for evaluating the risk of an adverse pregnancy outcome in a subject.

In some aspects, the present invention provides a method of evaluating the risk of an adverse pregnancy outcome in a subject comprising a) obtaining a sample from a pregnant subject; b) obtaining information regarding the level of Anti-Müllerian Hormone (AMH) in the sample, wherein if the subject does have an abnormal level of AMH as compared to a predetermined normal level the subject is more likely to have an adverse pregnancy outcome, and if the subject does not have an abnormal level of AMH the subject is less likely to have an adverse pregnancy outcome.

The sample may be any sample from a patient in which the AMH level may be assessed. In some embodiments, the sample may be a blood sample.

The sample may be obtained at any time during the pregnancy. For example, the sample may be obtained at or before 41 weeks after the subject's last monthly period. In some embodiments, the sample is obtained at or before 35, 30, 25, 20, 15, 10, or 5 weeks after the subject's last monthly period. In other embodiments, the sample is obtained between 4 and 41 weeks after the subject's last monthly period. In other embodiments, the sample is obtained between 10 and 25 weeks after the subject's last monthly period. In some embodiments, the sample is obtained at about 15 weeks after the subject's last monthly period. In some embodiments, the sample is obtained at about 10 weeks after the subject's last monthly period. In some embodiments, the sample is obtained between 11 and 15 weeks after the subject's last monthly period.

An abnormal level of AMH may be higher or lower than a predetermined level. A predetermined may be determined by any known method. In some embodiments, the abnormal level may be higher or lower than the predetermined level. The predetermined level may be a normal level or an abnormal level. In some embodiments, the predetermined normal level is based on a control. In some embodiments, the control is the level of AMH during pregnancy in a woman who had a normal obstetric outcome. In some embodiments, the control is the level of AMH during pregnancy in a woman who had an adverse obstetric outcome. In some embodiments, the abnormal level of AMH is higher than the level of AMH in the control. In some embodiments, the abnormal level of AMH is lower than the level of AMH in the control. In some embodiments, the abnormal level of AMH is the same as the level of AMH in the control. In some embodiments, the abnormal level of AMH is at least twice the level of the control, where the control is the level of AMH in a woman with a normal obstetric outcome.

In some embodiments, the measurement is repeated multiple times during the pregnancy. For example, a first sample may be obtained early in the pregnancy, e.g., between 4 and 15 weeks after the subject's last monthly period, and then a second sample may be obtained later in the pregnancy, e.g., between 15 and 41 weeks after the subject's last monthly period. In other embodiments, the first sample may be obtained between 4 and 20 weeks after the subject's last monthly period and the second sample may be obtained between 21 and 41 weeks after the subject's last monthly period.

Adverse pregnancy outcomes, or adverse obstetric outcomes, are well known in the art. Examples include, but are not limited to, preeclampsia, intrauterine growth restriction, preterm labor, premature rupture of the membrane, diabetes, and multiple gestation. In particular embodiments, the adverse pregnancy outcome is preterm delivery.

In other aspects, the present invention provides a method of evaluating the risk of an adverse pregnancy outcome in a subject comprising a) determining the level of AMH in a sample from a pregnant subject; and b) determining the risk of an adverse pregnancy outcome, wherein if the subject does have an abnormal level of AMH as compared to a predetermined normal level the subject is more likely to have an adverse pregnancy outcome, and if the subject does not have an abnormal level of AMH the subject is less likely to have an adverse pregnancy outcome.

The level of AMH in the sample may be determined by any appropriate method known to those of skill in the art. In some embodiments, the level of AMH in the sample is determined immunologically. In some embodiments, the level of AMH in the sample is determined by ELISA.

In other aspects, the present invention provides a method of evaluating the risk of an adverse pregnancy outcome in a subject comprising obtaining information regarding the level of AMH in a sample from a pregnant subject, wherein if the subject does have an abnormal level of AMH as compared to a predetermined normal level, the subject is more likely to have an adverse pregnancy outcome, and if the subject does not have an abnormal level of AMH, the subject is less likely to have an adverse pregnancy outcome.

In other aspects, the present invention provides a method of treating a subject who has been identified as at risk for an adverse pregnancy outcome comprising monitoring the patient in order to prevent an adverse pregnancy outcome, wherein the subject was identified as being at risk for an adverse pregnancy outcome due to an abnormal level of AMH as compared to a predetermined normal level.

In some aspects, the current invention provides a method of determining gender of a fetus. In some embodiments, the method comprises obtaining information regarding the level of AMH in a sample from a pregnant subject, wherein if the subject has a level of AMH which is the same as or higher than a control the fetus is a male. In some embodiments, the method comprises obtaining information regarding the level of AMH in a sample from a pregnant subject, wherein if the subject has a level of AMH which is the same as or lower than a control the fetus is a female.

In some aspects, the current invention provides a method of determining gender of a fetus. In some embodiments, the method comprises determining the level of AMH in a sample from a pregnant subject, wherein if the subject has a level of AMH which is the same as or higher than a predetermined level or control, the fetus is a male. In some embodiments, the method comprises determining the level of AMH in a sample from a pregnant subject, wherein if the subject has a level of AMH which is the same as or lower than a predetermined level or control, the fetus is a female.

In some embodiments, the control is the level of AMH in a woman carrying a female fetus. In such embodiments, a level of AMH in a sample which is higher than the predetermined level or control indicates a male fetus and a level of AMH in a sample which is the same as the predetermined level or control is a female fetus. In some embodiments, the control is the level of AMH in a woman carrying a male fetus. In such embodiments, a level of AMH in a sample which is lower than the predetermined level or control indicates a female fetus and a level of AMH in a sample which is the same as the predetermined level or control is a male fetus.

The sample may be obtained at any time during pregnancy. In some embodiments, the sample is obtained at or before 15 weeks after the subject's last monthly period and at or after 11 weeks after the subject's last monthly period. In some embodiments, the measurement is repeated multiple times during the pregnancy. For example, a first sample may be obtained early in the pregnancy, e.g., at or between 11 and 13 weeks after the subject's last monthly period, and then a second sample may be obtained later in the pregnancy, e.g., at or between 13 and 15 weeks after the subject's last monthly period.

In some embodiments, the subject is a human subject. In other embodiments, the subject is a non-human subject, such as a bovine, equine, or canine subject.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The term “therapeutically effective” as used herein refers to an amount of cells and/or therapeutic composition (such as a therapeutic polynucleotide and/or therapeutic polypeptide) that is employed in methods of the present invention to achieve a therapeutic effect, such as wherein at least one symptom of a condition being treated is at least ameliorated.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Scatter plot of all AMH samples from women who went on to have normal obstetric outcomes, including term deliveries.

FIG. 2 Scatter plot of all AMH samples from women who went on to have a preterm delivery.

FIG. 3 Mean AMH level by gestational age stratified by preterm delivery. AMH levels are adjusted for maternal age, multiple measures and total protein. The difference in AMH levels in the 11-15 week window are significant (p<0.05).

FIG. 4 Mean AMH level by gestational age at the time of blood draw. AMH levels are adjusted for maternal age, multiple measures and total protein. The decline over time is significant (p<0.0001).

FIGS. 5A-B FIG. 5A: Mean AMH level corrected for maternal age, total protein and multiple measures. AMH for all gestational ages stratified by fetal sex. Difference is not significant. FIG. 5B: Mean AMH levels corrected for maternal age, total protein and multiple measures. AMH stratified by fetal sex and gestational week of blood draw. Overall p value of <0.0001 allows for examination of individual strata. Significant difference seen between early and late gestational ages as well as for differences between sex of fetus at 11-15 weeks gestation.

### DESCRIPTION OF THE ILLUSTRATIVE EMBODIMENTS

The present invention provides methods for evaluating the risk of an adverse pregnancy outcome in a subject by determining the level of Anti-Müllerian Hormone (AMH) in a sample. The sample may be taken during pregnancy, for example early in the pregnancy. Based on the results of this test, women are classified into high risk or low risk categories. For example, if the subject has an abnormal level of AMH as compared to a predetermined normal level, the subject is more likely to have an adverse pregnancy outcome. Conversely, if the subject does not have an abnormal level of AMH, the subject is less likely to have an adverse pregnancy outcome. The predetermined normal level may be determined, for example, by comparison to women who have had normal obstetric outcomes. The high risk category would identify women who require more intensive monitoring. In some embodiments, an abnormal level of AMH may indicate a need for additional testing. In some embodiments, a sample taken early in the pregnancy and a sample taken later in the pregnancy may be analyzed, where the results are compared to determine whether or not the subject should be classified as high risk or low risk.

An abnormal level of AMH may be higher or lower than a normal level. In some embodiments, the normal level of AMH is determined by comparison to the level of AMH in a control. In some embodiments, the control is a subject who had a normal obstetric outcome. In other embodiments, the control is a subject who has had an adverse obstetric outcome. In other embodiments, the normal level of AMH is a standard level that has been predetermined. In some embodiments, the rate of decline of AMH during pregnancy is different for a subject who is predicted to experience an adverse pregnancy outcome. For example, the level of AMH may start at a normal level but the decline would occur later or earlier in the pregnancy for a subject who is predicted to experience an adverse pregnancy outcome.

In some aspects, this invention provides methods of identifying subjects that have a high risk of experiencing an adverse obstetric outcome. Once a subject is identified as at high risk of an adverse obstetric outcome, close monitoring of these patients and possible therapeutic intervention may be applied to help prevent preterm birth.

Benefits of this method include the fact that the test is minimally invasive and predicts adverse outcomes at a time when interventions would be highly effective. This method may also be used as a marker for success of treatments used to decrease risk for a known adverse outcome, such as use of a medication to decrease the risk for preterm labor.

### A. ADVERSE PREGNANCY OUTCOMES

In some aspects, this method relates to evaluating the risk of an adverse pregnancy outcome in a subject. Adverse pregnancy outcomes, or adverse obstetric outcomes, are well known in the art. Examples include, but are not limited to, preeclampsia, intrauterine growth restriction, preterm labor, premature rupture of the membrane, diabetes, and multiple gestation.

1. Preterm Labor

A full-term pregnancy lasts about 40 weeks. Preterm labor refers to contractions that begin to open the cervix before week 37. Preterm delivery refers to a delivery of a child after 20 weeks and before 37 weeks gestation. Pre-term labor may result in the birth of a premature baby. However, labor often can be stopped to allow the baby more time to grow and develop in the uterus. Premature labor treatments include bed rest, fluids given intravenously, medications to relax the uterus, and use of intramuscular progesterone in early pregnancy as a preventive measure.

Often, the specific cause of preterm labor isn't clear. If preterm labor can't be stopped, the baby will be born too soon, and the earlier preterm birth happens, the greater the risks for the baby, including low birth weight, breathing difficulties, underdeveloped organs and potentially life-threatening infections. Children who are born prematurely also have a higher risk of learning disabilities, developmental disabilities and behavior problems.

2. Pre-Eclampsia

Preeclampsia is a condition of pregnancy marked by high blood pressure and excess protein in urine after 20 weeks of pregnancy. Left untreated, preeclampsia can lead to serious, even fatal, complications for both the mother and the baby.

If a subject has preeclampsia, the only cure is delivery of the baby. If a subject is diagnosed with preeclampsia too early in the pregnancy for delivery to be an option, the doctor needs to allow the baby more time to mature, without putting the mother or the baby at risk of serious complications.

Preeclampsia can develop gradually but often attacks suddenly, after 20 weeks of pregnancy and may range from mild to severe. If the subject's blood pressure was normal before the pregnancy, signs and symptoms of preeclampsia may include high blood pressure (hypertension), excess protein in the urine (proteinuria), severe headaches, changes in vision, including temporary loss of vision, blurred vision or light sensitivity, upper abdominal pain, usually under the subject's ribs on the right side, nausea or vomiting, dizziness, decreased urine output, or sudden weight gain, typically more than 2 pounds a week.

Researchers have yet to determine what causes preeclampsia. Possible causes may include insufficient blood flow to the uterus, damage to the blood vessels, a problem with the immune system, or poor diet.

3. Intrauterine Growth Restriction

Intrauterine growth restriction (IUGR) refers to the poor growth of a baby while in the mother's womb during pregnancy. Specifically, it means the developing baby weights less than 90% of other babies at the same gestational age.

Many different things can lead to IUGR. An unborn baby may not get enough oxygen and nutrition from the placenta during pregnancy because of various reasons, including, for example, high altitudes, multiple pregnancy (twins, triplets, etc.), placenta problems, and preeclampsia or eclampsia. Infections during pregnancy that affect the developing baby, such as rubella, cytomegalovirus, toxoplasmosis, and syphilis may also affect the weight of the developing baby.

Congenital or chromosomal abnormalities are often associated with below-normal weight. IUGR also increases the risk that the baby will die inside the womb before birth. If the doctor thinks the subject might have IUGR, the subject will be closely monitored with several pregnancy ultrasounds to measure the baby's growth, movements, blood flow, and fluid around the baby. Non-stress testing will also be done. Depending on the results of these tests, delivery may be necessary.

4. Premature Rupture of the Membrane

Premature rupture of membranes (PROM) is a condition that occurs in pregnancy when there is rupture of the membranes (rupture of the amniotic sac and chorion) more than an hour before the onset of labor. Premature rupture of membranes (PROM) refers to a patient who is beyond 37 weeks' gestation and has presented with rupture of membranes (ROM) prior to the onset of labor. Preterm premature rupture of membranes (PPROM) is ROM prior to 37 weeks' gestation.

Eighty-five percent of neonatal morbidity and mortality is a result of prematurity. PPROM is associated with 30-40% of preterm deliveries, is the leading identifiable cause of preterm delivery, complicates 3% of all pregnancies and occurs in approximately 150,000 pregnancies yearly in the United States. When PPROM occurs long before term, significant risks of morbidity and mortality are present for both the fetus and the mother.

Risk factors for PROM can be a bacterial infection, smoking, or anatomic defect in the structure of the amniotic sac, uterus, or cervix. In some cases, the rupture can spontaneously heal, but in most cases of PPROM, labor begins within 48 hours. When this occurs, it is necessary that the mother receives treatment to avoid possible infection in the newborn.

5. Gestational Diabetes

Gestational diabetes (or gestational diabetes mellitus (GDM)) is a condition in which women without previously diagnosed diabetes exhibit high blood glucose levels during pregnancy (especially during third trimester of pregnancy).

Gestational diabetes generally has few symptoms and it is most commonly diagnosed by screening during pregnancy. Diagnostic tests detect inappropriately high levels of glucose in blood samples. Gestational diabetes affects 3-10% of pregnancies, depending on the population studied.

Babies born to mothers with gestational diabetes are typically at increased risk of problems such as being large for gestational age (which may lead to delivery complications), low blood sugar, and jaundice. Gestational diabetes is a treatable condition and women who have adequate control of glucose levels can effectively decrease these risks.

Women with gestational diabetes are at increased risk of developing type 2 diabetes mellitus (or, very rarely, latent autoimmune diabetes or Type 1) after pregnancy, as well as having a higher incidence of pre-eclampsia and Caesarean section; their offspring are prone to developing childhood obesity, with type 2 diabetes later in life. Most patients are treated only with diet modification and moderate exercise but some take anti-diabetic drugs, including insulin.

6. Multiple Gestation

A multiple birth occurs when more than one fetus is carried to term in a single pregnancy. Different names for multiple births are used, depending on the number of offspring. Common multiples are two and three, known as twins and triplets. These and other multiple births occur to varying degrees in most animal species, although the term is most applicable to placental species.

Babies born from multiple-birth pregnancies are more likely to result in premature birth than those from single pregnancies. 51% of twins and 91% of triplets are born preterm, compared to 9.4% in singletons. 14% of twins and 41% of triplets are even born very preterm, compared to 1.7% in singletons. The preterm births also result in multiples tending to have a lower birth weight compared to singletons.

### B. ANTI-MÜLLERIAN HORMONE

Anti-Müllerian Hormone (AMH), also known as Müllerian-inhibiting substance, is a dimeric glycoprotein that belongs to the transforming growth factor-B family (Cate et al., 1986). AMH is present in fish, reptiles, birds, marsupials, and placental mammals. It is involved in the regression of the Müllerian ducts during male fetal development and is expressed in Sertoli cells from testicular differentiation up to puberty. In females, AMH is exclusively produced by granulosa cells of preantral (primary and secondary) and small antral follicles from birth up to menopause. Production of AMH starts after follicles differentiate from the primordial to the primary stage and it continues until the follicles have reached the antral stages. The number of the small antral follicles is related to the size of the primordial follicular pool. With the decrease in the number of the antral follicles with age, AMH production appears to become diminished, and it invariably will become undetectable at menopause.

Several studies in which AMH was absent or overexpressed indicated loss of inhibition (if absent) or an excessive inhibitory effect (if overexpressed) of AMH on growing follicles (Lyet et al., 1995; Durlinger et al., 1999). AMH is detected in serum from women of reproductive age and its levels do not vary with the menstrual cycle (LaMarca et al, 2009). Serum AMH levels also have been shown to decrease slowly over time as the primordial follicular pool declines in normo-ovualtory women (de Vet et al., 2002), and to correlate with age and the number of antral follicles. Therefore, AMH might represent a sensitive marker for ovarian aging (Fanchin et al., 2003). Indeed, it has been shown that poor response during in vitro fertilization (IVF), indicative of a diminished ovarian reserve (Beckers et al., 2002), is associated with reduced baseline serum AMH concentrations (Seifer et al., 2002). An inverse relationship has been observed between estradiol (E₂) and AMH plasma levels (Fanchin et al., 2003), suggesting that E₂ may have a negative role on AMH production, or vice versa.

In recent years, it has been established that plasma AMH levels, which correlate with the number of antral and preantral follicles in mice, as with humans, can be used for assessing ovarian reserve. AMH also has been proposed as a surrogate marker of the antral follicular count (AFC) in polycystic ovary syndrome. Mounting evidence also indicates that AMH levels, which reflect the size of the cohort of recruitable follicles, also predict the magnitude of the ovarian response to controlled ovarian hyperstimulation (COH). As the number of preantral and antral follicles directly reflects the size of the cohort of primordial follicles, AMH levels have been proposed as a marker of ovarian reserve.

The current paradigm holds that AMH is independent of outside hormonal influences and remains constant from childhood until ovarian function is lost at menopause (LaMarca et al., 2009; Hagen et al., 2010; LaMarca et al., 2004; Franchin et al., 2005; Streuli et al., 2009; Streuli et al., 2008). AMH is secreted by growing ovarian follicles and is the only source of AMH in females. AMH levels are, therefore, a direct reflection of the size of the growing follicular pool (van Rooij et al., 2005; Broekmans et al., 2006; Kwee et al., 2008). A decline in AMH levels would indicate a loss of this growing follicular pool (van Rooij et al., 2005; Broekmans et al., 2006; Kwee et al., 2008) and fewer follicles available for ovulation. From an evolutionary standpoint, active inhibition of follicular development during gestation is one important method to prevent a second, concurrent pregnancy. A rapid decline in AMH levels in a very short window would indicate that AMH is being actively suppressed, as this pattern is not consistent with the pattern of passive AMH decline seen after loss of follicle-stimulating hormone (FSH) stimulation (Partridge et al., 2010; Andersen and Byskov, 2006; Anderson et al., 2006). However, active suppression of AMH during pregnancy has only recently been described (Nelson et al., 2010; Li et al, 2010; Seifer et al., 2007). In fact, La Marca et al. (2006), reported that AMH levels did not change throughout pregnancy while Li et al., (2010) and Seifer et al., (2010) mentioned that the decline occurred between 13-15 weeks.

### C. DETECTION OF AMH

1. Protein Detection

In some embodiments, the present invention concerns determining the level of one or more hormones or proteins in a sample. In certain embodiments, the present invention concerns determining the level of AMH, a protein hormone, in a sample. In other embodiments, the present invention concerns determining the level of multiple hormones in a sample.

As used herein, a “protein,” “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

a. Immunodetection Methods

As discussed, in some embodiments, the present invention concerns immunodetection methods for quantifying, binding, purifying, removing, and/or otherwise detecting biological components, such as AMH. Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis and Galand (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

b. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

c. Antibodies

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (monoclonal antibodies) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is also used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).

The methods for generating monoclonal antibodies (monoclonal antibodies) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur within a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or down-regulate suppressor cell activity. Such BRMs include, but are not limited to, Cimetidine (CIM; 1200 mg/d) (Smith/Kline, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

Monoclonal antibodies may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonal antibodies. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

d. Protein Arrays

Protein array technology is discussed in detail in Pandey and Mann (2000) and MacBeath and Schreiber (2000), each of which is herein specifically incorporated by reference.

These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate an amount of a proteins in a sample, such as AMH.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

2. Nucleic Acid Detection

Detection of nucleic acids encoding AMH are also encompassed by the invention. In certain embodiments, the present invention concerns determining the level of AMH expression by determining the level of gene expression. Generally, the present invention concerns polynucleotides and oligonucleotides, isolatable from cells, that are free from total genomic DNA and that are capable of expressing all or part of a protein or polypeptide. The polynucleotides or oligonucleotides may be identical or complementary to all or part of a nucleic acid sequence encoding an AMH amino acid sequence. These nucleic acids may be used directly or indirectly to assess, evaluate, quantify, or determine AMH expression.

As used in this application, the term “AMH polynucleotide” refers to a AMH-encoding nucleic acid molecule that has been isolated essentially or substantially free of total genomic nucleic acid to permit hybridization and amplification, but is not limited to such. Therefore, a “polynucleotide encoding AMH” refers to a DNA segment that contains wild-type, mutant, or polymorphic AMH polypeptide-coding sequences isolated away from, or purified free from, total mammalian or human genomic DNA. An AMH oligonucleotide refers to a nucleic acid molecule that is complementary or identical to at least 5 contiguous nucleotides of an AMH-encoding sequence, which is the cDNA sequence encoding AMH.

It also is contemplated that a particular polypeptide from a given species may be represented by natural variants that have slightly different nucleic acid sequences but, nonetheless, encode the same protein.

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including such sequences from AMH encoding sequences.

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a AMH-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

c. Chip Technologies

Chip-based DNA technologies such as those described by Hacia et al. (1996) and Shoemaker et al. (1996) may also be used. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of AMH with respect to diagnostic, as well as preventative and treatment methods of the invention.

d. Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Two hundred and fifty five samples were acquired from 191 women that were randomly distributed across all gestational ages (range 5.6 to 41.1 weeks). Outcome information was available on 243 (94%) of these women; 197 had term deliveries, and 46 had preterm deliveries. Thirty-five of the samples were collected prior to 10 weeks, 44 between 11-15 weeks, 62 between 16 and 20 weeks, 20 between 21 and 25 weeks, 29 between 26 and 30 weeks, 21 between 31 and 35 weeks, 29 between 36 and 40 weeks, and 16 at greater than 40 weeks. Sixty-four women were sampled more than one time in the pregnancy.

Results from the full cohort as well as the two sub-cohorts are listed in Table 1. The analysis is adjusted for multiple measures and maternal age. Significance was found in all comparisons. The mean gestational age for both normal pregnancy and preterm subjects is shown in the legend of the table.

TABLE 1
Mean AMH Levels adjusted for maternal age and multiple measures

| | Male | Female | |
|---|---|---|---|
| All gestational ages* | 0.96 ± 0.07 | 0.73 ± 0.04 | 0.10 |

| Week of blood draw* | Entire Cohort | N | Male | N | Female | p |
|---|---|---|---|---|---|---|
| <10 weeks | 1.48 ± 0.08 | 12 | 1.66 ± 0.12 | 9 | 1.66 ± 0.14 | 0.64 |
| 11-15 weeks | 1.23 ± 0.06 | 17 | 1.80 ± 0.08 | 23 | 0.87 ± 0.12 | 0.0015 |
| 16-20 weeks | 0.82 ± 0.06 | 18 | 0.89 ± 0.09 | 21 | 0.88 ± 0.08 | 0.64 |
| 21-25 weeks | 0.87 ± 0.11 | 4 | 1.43 ± 0.05 | 8 | 0.73 ± 0.15 | 0.29 |
| 26-30 weeks | 0.70 ± 0.09 | 9 | 1.02 ± 0.10 | 8 | 0.64 ± 0.08 | 0.29 |
| 31-35 weeks | 0.40 ± 0.08 | 4 | 0.42 ± 0.13 | 5 | 0.29 ± 0.12 | 0.13 |
| 36-40 weeks | 0.39 ± 0.08 | 10 | 0.41 ± 0.09 | 9 | 0.51 ± 0.07 | 0.13 |
| >40 weeks | 0.26 ± 0.10 | 8 | 0.30 ± 0.10 | 5 | 0.20 ± 0.06 | 0.5 |

*Mean ± SEM

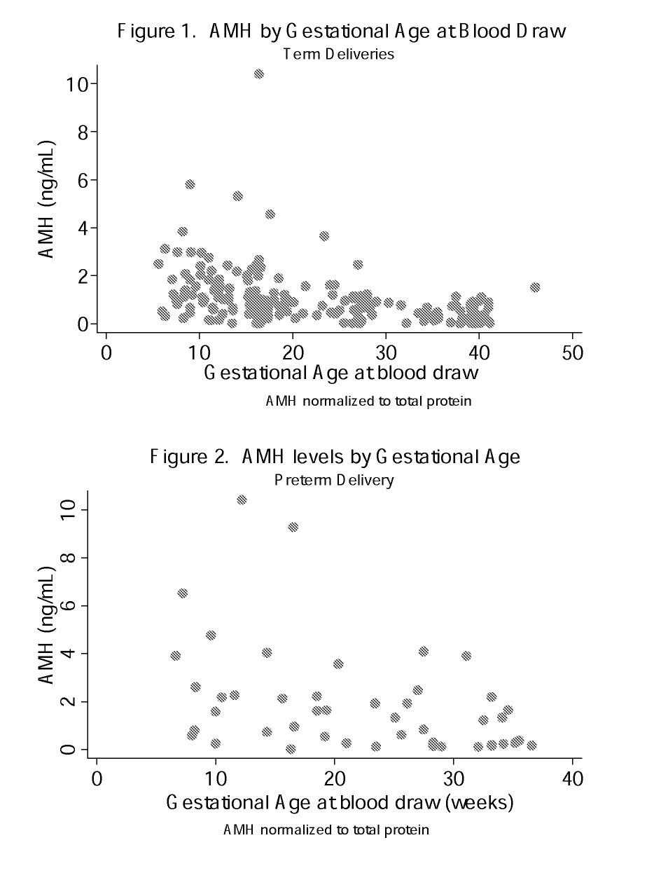

FIG. 1 shows the scatter plot of all AMH samples from women who went on to have normal obstetric outcomes. FIG. 2 shows the scatter plot of all AMH samples from women who went on to have a preterm delivery. This pattern demonstrated that the drop in AMH around 15 weeks gestation. However, in women who have had a preterm delivery do not show the drop in AMH until much later in pregnancy and are more likely to have an elevated AMH levels throughout the entire pregnancy.

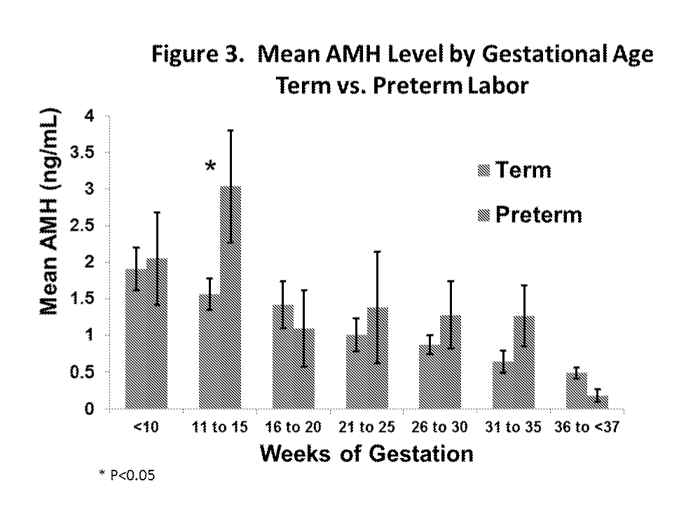

These data suggest that there was a decline in AMH associated with normal obstetric outcomes, and that it occurred around 11-15 weeks gestation. This decline was not seen in women with preterm deliveries, and the inventors have theorized that this is due to abnormal feto-placental signaling (FIG. 3). Some of the pregnancies were twin gestation, but this was controlled for in the model and these women were stratified in the model according to the obstetric outcome.

Finally, the Beckman Coulter Generation II immunoassay was used to analyze the plasma samples. This immunoassay is available for research purposes only and uses a completely different set of antibodies that the previous generation immunoassay. Therefore, the analysis was repeated using the first generation AMH assay, and found similar pattern of results (results not shown).

This work suggests that AMH, a marker released from growing ovarian follicles, does not decline at the same rate in women who develop preterm labor versus those with normal pregnancy outcomes, indicating that the ovary may not be downregulated appropriately during pregnancy. This may be related to problems with feto-placental signaling and loss of the mediator responsible for this downregulation. Thus, AMH may be a useful surrogate marker for improper feto-placental signaling that may lead to preterm delivery.

### Example 2

Methods:

167 samples from 112 women were obtained with gestational ages (GA) between 5.6-41.0 weeks. 82 samples from 54 women with outcome data were also analyzed. AMH was measured using AMH GenII Immunoassay (Beckman Coulter). AOO included preterm labor (PTL), premature rupture of membrane (PPROM), and preeclampsia/intrauterine growth restriction (pre-e/IUGR). Multivariate regression was used for analysis, controlling for multiple measures and maternal age.

Results:

AMH measurements were grouped by trimester. Mean AMH levels in the entire dataset declined significantly between the 1st and 3rd trimesters (p<0.05).

### Example 3

One hundred and thirty two samples were obtained from women and obstetrics outcomes were analyzed. Women were divided into two groups: those that delivered after 37 weeks (normal outcome) and those who delivered prior to 37 weeks (preterm labor). AMH levels were measured and results were analyzed. Women in the two groups were similar with the exception of the time of delivery (38 w 1 day in normal outcomes, 34 w 4 days in the preterm labor outcomes) and the average AMH level for all gestational ages. In women who had preterm labor, AMH levels were significantly higher until 20 weeks of pregnancy. AMH levels after 20 weeks did not differ between the two groups. See Table 2. This may indicate that high AMH levels prior to 20 weeks of pregnancy can predict women who will go on to experience preterm labor, and would be classified as high risk. Close monitoring of these patients and possible therapeutic intervention may be applied to help prevent preterm birth.

TABLE 2

| | Normal Outcome | Preterm Labor | p |
|---|---|---|---|
| Total in Sample | 118 | 14 | |
| Age Mean (SD) | 29.6 (5.1) | 29.1 (3.9) | 0.74 |
| AMH - entire | 1.5 (1.4) | 3.4 (4.1) | 0.0004 |
| pregnancy Mean (SD) | | | |
| Gestational age at | 38 w 1 d | 34 w 4 d | 0.0001 |
| delivery Mean (SD) | (2 w 6 d) | (1 w 6 d) | |
| Mean AMH | 1.9 | 13.9 | ** |
| Weeks 11-15 | (n = 25) | (n = 1) | |
| Mean AMH (SD) | 1.6 (0.3) | 5.8 | 0.0033 |
| Weeks 16-20 | (n = 21) | (n = 2) | |
| Mean AMH (SD) | 2.2 (1.6) | 0.2 (0.2) | 0.13 |
| Weeks 21-25 | (n = 9) | (n = 2) | |
| Mean AMH (SD) | 1.3 (1.4) | 0.5 | 0.31 |
| Weeks 26-30 | (n = 24) | (n = 4) | |
| Mean AMH (SD) | 1.5 (1.9) | 2.1 (0.5) | 0.7 |
| Weeks 31-35 | (n = 6) | (n = 2) | |

### Example 4

### Methods

Sample Collection.

De-identified maternal plasma samples were obtained from an Institutional Review Board approved tissue repository, the University of Iowa Maternal Fetal Tissue Bank (MFTB). For this study, tissue bank samples from women at all gestational ages were matched with their corresponding de-identified outcome data and were available for use. Inclusion criteria for our study were: maternal age of at least 18 years and pregnancy ending in term deliveries (inductions, spontaneous vaginal deliveries, and c-sections) without complications. Women were excluded from if they were positive for Hepatitis C or HIV, or were diagnosed with multiple gestation, preterm delivery, or other abnormal pregnancy outcomes.

AMH Measurement.

Anti-mullerian hormone (AMH) was measured in each plasma sample by Enzyme Linked Immunosorbant Assay (ELISA) using the AMH Gen II Immunoassay (Beckman Coulter, Chaska, Minn.). Samples were batched and run in duplicate. The bicinchoninic acid assay (Pierce) was used to measure total protein in plasma. AMH levels were normalized to total protein. Clinical data available for the samples included maternal age, infant sex, and gestational age at the time of each blood draw.

Statistical Analysis.

Statistical analysis was completed using Stata 11.2 (College Station Tex.). Univariate and bivariate comparisons were completed as required to assess distribution of variables. Student t-test and Pearson's chi-square were used as appropriate. Non-linear continuous variables were log-transformed prior to adding to the model or transformed into categorical variables as appropriate. Logistic regression modeling of the mean AMH levels and AMH levels by gestational age were compared between women with male and female fetuses adjusting for maternal age and multiple measures initially grouped over the entire pregnancy. The results were then stratified by gestational week at blood draw.

### Results

There were 170 samples from 131 women (61 with boys and 70 with girls). Because individual could be sampled multiple times, there were a total of 82 samples from boys and 87 samples from girls. Crude analysis showed no differences in the average age, gestational age at delivery, or number of samples from each gestational category by fetal sex (Table 3). Gestational age and AMH levels were not normally distributed. Therefore, gestational age was transformed into a categorical variable using the following groups: <10 weeks, 11-15 weeks, 16-20 weeks, 21-25 weeks, 26-30 weeks, 31-35 weeks, 36-40 weeks, and >40 weeks. After standardizing AMH levels to total blood protein levels, the levels were log-transformed prior to inclusion in the model.

TABLE 3
Crude Comparisons of the Entire Cohort and by Sex of the Fetus

| | Entire cohort N = 71 | Male n = 78 | Female n = 76 |
|---|---|---|---|
| Age* | 30 ± 3 | 30.4 ± 5.1 | 30.2 ± 4.8 |
| Gestational age | 39 w 4 d ± 7 d | 39 w 2 d ± 7 d | 39 w 2 d ± 7 d |
| at delivery* | | | |
| <10 wk | 21 | 12 | 9 |
| 11-15 wk | 40 | 17 | 23 |
| 16-20 wk | 39 | 18 | 21 |
| 21-25 wk | 12 | 4 | 8 |
| 26-30 wk | 17 | 9 | 8 |
| 31-35 wk | 9 | 4 | 5 |
| 36-40 wk | 19 | 10 | 9 |
| >40 | 13 | 8 | 5 |

*Mean ± SD, p > 0.05
**Frequency, p > 0.05 for differences in frequency between males and females

TABLE 4
Mean AMH Levels adjusted for maternal age and multiple measures

| | Male | Female | |
|---|---|---|---|
| All gestational ages* | 0.96 ± 0.07 | 0.73 ± 0.04 | 0.1 |

| Week of blood draw* | Entire Cohort | N | Male | N | Female | p |
|---|---|---|---|---|---|---|
| <10 weeks | 1.48 ± 0.08 | 12 | 1.66 ± 0.12 | 9 | 1.66 ± 0.14 | 0.64 |
| 11-15 weeks | 1.23 ± 0.06 | 17 | 1.80 ± 0.08 | 23 | 0.87 ± 0.12 | 0.0015 |
| 16-20 weeks | 0.82 ± 0.06 | 18 | 0.89 ± 0.09 | 21 | 0.88 ± 0.08 | 0.64 |
| 21-25 weeks | 0.87 ± 0.11 | 4 | 1.43 ± 0.05 | 8 | 0.73 ± 0.15 | 0.29 |
| 26-30 weeks | 0.70 ± 0.09 | 9 | 1.02 ± 0.10 | 8 | 0.64 ± 0.08 | 0.29 |
| 31-35 weeks | 0.40 ± 0.08 | 4 | 0.42 ± 0.13 | 5 | 0.29 ± 0.12 | 0.13 |
| 36-40 weeks | 0.39 ± 0.08 | 10 | 0.41 ± 0.09 | 9 | 0.51 ± 0.07 | 0.13 |
| >40 weeks | 0.26 ± 0.10 | 8 | 0.30 ± 0.10 | 5 | 0.20 ± 0.06 | 0.5 |

*Mean ± SEM

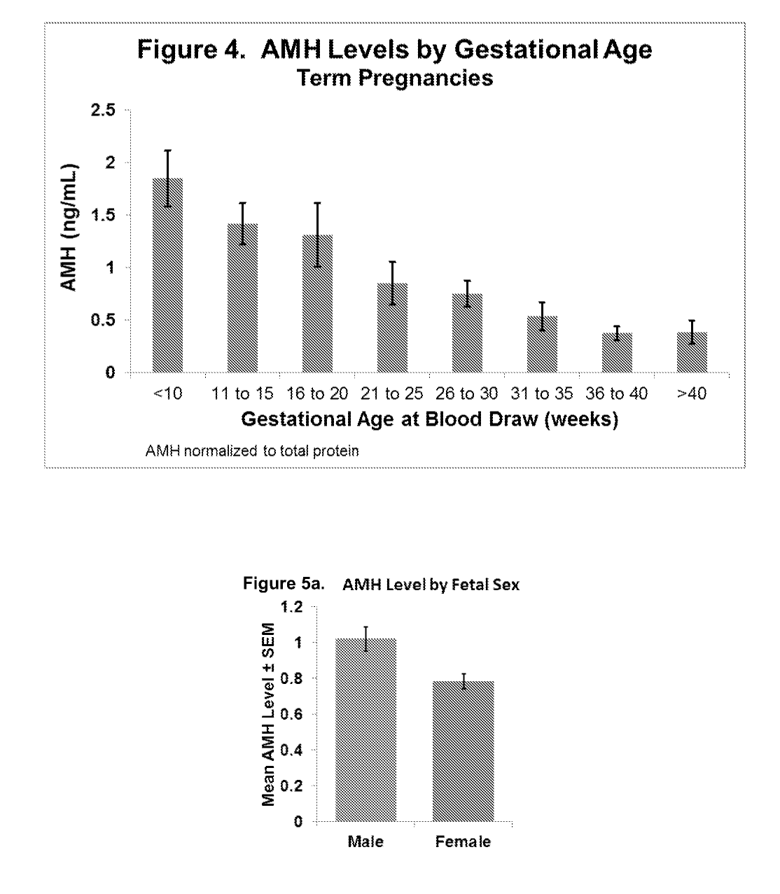

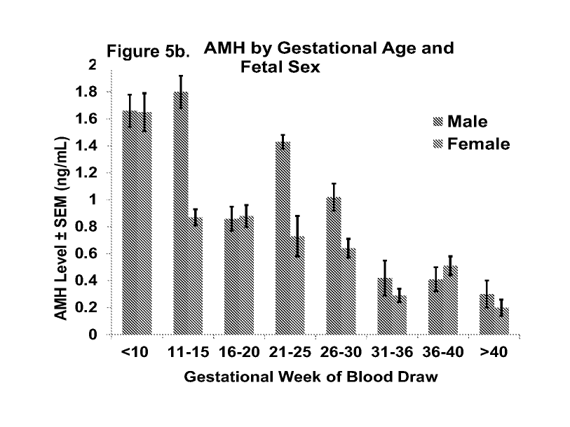

Mean AMH levels were compared, first by gestational age category and then by fetal sex, averaged over the entire pregnancy. When stratified by gestational age at the time of blood draw, AMH levels showed a decline throughout pregnancy, and the differences between AMH levels at the beginning of pregnancy and at term were statistically significant. (p<0.0001) (FIG. 4). When stratified by fetal sex alone, there the difference between mean AMH levels was not statistical significance (males: 0.96±0.07, females 0.73±0.04, p=0.10) (FIG. 5A). However, after stratifying for both gestational age and fetal sex, male and female AMH levels differed significantly in the 11-15 weeks window of gestational age, with mean AMH levels being significantly higher in women with male fetuses compared to women with female fetuses (2.0±0.35 ng/mL vs. 0.94±0.20 ng/mL) (FIG. 5B).

A drop in AMH between 11-15 weeks gestation confirm a declining AMH in pregnancy. AMH levels are, however, different in the 11-15 week window based on the sex of the fetus. In addition, it appears that onset of the decline lags in women with a male fetus versus a female fetus, resulting in overall higher levels in women carrying males. Outside of this limited period, AMH levels are similar and when averaged over the entire pregnancy, there is no significant difference in AMH levels based on fetal sex, in that there is a significant fall in AMH in pregnancy regardless of the fetal sex. In contrast, AMH levels averaged over the entire pregnancy are not different between males and females.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,324,633
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,571,639
U.S. Pat. No. 5,580,726
U.S. Pat. No. 5,580,732
U.S. Pat. No. 5,593,839
U.S. Pat. No. 5,599,672
U.S. Pat. No. 5,599,695
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,744,305
U.S. Pat. No. 5,800,992
U.S. Pat. No. 5,807,522
U.S. Pat. No. 5,830,645
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,876,932
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Patent Publin. 2008/0009439
Andersen and Byskov, J. Clin. Endocrinol. Metab., 91(10):4064-4069, 2006.
Anderson et al., Clin. Endocrinol., 64(6):715-716, 2006.
Beckers et al., Fertil Steril., 78(2):291-297, 2002.
Bellus, J. Macromol. Sci. Pure Appl. Chem., A31(1): 1355-1376, 1994.
Broekmans et al., Hum. Reprod., 12(6):685-718, 2006.
Cate et al., Cell, 45(5):685-698, 1986.
De Jager et al., Semin. Nucl. Med., 23(2):165-179, 1993.
de Vet et al., Fertil Steril., 77(2):357-62, 2002.
Doolittle et al., Methods Mol. Biol., 109:215-237, 1999.
Durlinger et al., Endocrinology, 140(12):5789-5796, 1999.
EP 329 822
EP 373 203
EP 785 280
EP 799 897
Fanchin et al., Hum Reprod., 18(2):328-32, 2003.
Fanchin et al., Hum Reprod., 20(4):923-927, 2005.
Fodor et al., Science, 251:767-773, 1991.
Frohman, In: PCR Protocols: A Guide To Methods And Applications, Academic Press, N.Y., 1990.
GB Appln. 2 202 328
Gulbis and Galand, Hum. Pathol., 24(12):1271-1285, 1993.
Hacia et al., Nature Genet., 14:441-449, 1996.
Hagen et al., J. Clin. Endocrinol. Metab., 95(11):5003-5010, 2010.
Harlow and Lane, In: Antibodies: A Laboratory Manual, Cold Spring Harbor Laboratory, 1988.
Heron et al., Pediatrics, 125(1):4-15, 2007.
Innis et al., Proc. Natl. Acad. Sci. USA, 85(24):9436-9440, 1988.
Kwee et al., Fertil. Steril., 90(3):737-743, 2008.
Kwoh et al., Proc. Natl. Acad. Sci. USA, 86:1173, 1989.
LaMarca et al., Hum. Reprod., 19(12):2738-2741, 2004.
LaMarca et al, Hum. Repro., 24(9):2264-2275, 2009.
Lyet et al., Biol. Reprod., 52(2):444-454, 1995.
MacBeath and Schreiber, Science, 289(5485):1760-1763, 2000.
Nakamura et al., In: Handbook of Experimental Immunology (4^{th} Ed.), Weir et al. (Eds), 1:27, Blackwell Scientific Publ., Oxford, 1987.
Nelson et al., Fertil. Steril., 93(4):1356-1358, 2010.
Ohara et al., Proc. Natl. Acad. Sci. USA, 86:5673-5677, 1989.
Pandey and Mann, Nature, 405(6788):837-846, 2000.
Partridge et al., Fertil. Steril., 94(2):638-644, 2010.
PCT Appln. PCT/87/00880
PCT Appln. PCT/89/01025
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100448A1
PCT Appln. WO 031/00012
PCT Appln. WO 04/020085
PCT Appln. WO 04/027093
PCT Appln. WO 88/10315
PCT Appln. WO 89/06700
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/23256
PCT Appln. WO 99/35505
PCT Appln. WO 99/36760
Pease et al., Proc. Natl. Acad. Sci. USA, 91:5022-5026, 1994.
Sambrook and Russell, Molecular Cloning: A Laboratory Manual 3^{rd} Ed., Cold Spring Harbor Laboratory Press, 2001.
Savitz, Am. J. Epidemiol., 168(9):990-992, 2008.
Seifer et al., Fertil Steril., 77(3):468-471, 2002.
Shoemaker et al., Nature Genetics, 14:450-456, 1996.
Silasi et al., Obstet. Gynecol. Clin. North Am., 37(2):239-253, 2010.
Streuli et al., Fertil. Steril., 90(2):395-400, 2008.
Streuli et al., Fertil. Steril., 91(1):226-230, 2009.
UK 8 803 000
Van Rooij et al., Fertil. Steril., 83(4):979-987, 2005.
Walker et al., Proc. Natl. Acad. Sci. USA, 89:392-396, 1992.
Wang et al., Physiology, 24:147-158, 2009.

1-57. (canceled)

58. A method of evaluating the risk of an adverse pregnancy outcome in a subject, comprising:
obtaining information regarding the level of Anti-Mullerian Hormone (AMH) in a sample obtained from a pregnant subject,
wherein if the subject has an abnormal level of AMH as compared to a control, the subject is more likely than the control to have an adverse pregnancy outcome, and if the subject does not have an abnormal level of AMH as compared to a control, the subject is less likely than the control to have an adverse pregnancy outcome.

59. The method of claim 58, wherein the information regarding the level of AMH in the sample is obtained by immunologically determining the level of AMH in the sample.

60. The method of claim 58, wherein the sample is a blood sample.

61. The method of claim 58, wherein the sample is obtained at or before 25 weeks, or at or before 20 weeks, or at or before 15 weeks, or at or before 10 weeks after the subject's last monthly period.

62. The method of claim 58, wherein the sample is obtained between 4 and 41 weeks after the subject's last monthly period.

63. The method of claim 62, wherein the sample is obtained between 10 and 25 weeks after the subject's last monthly period.

64. The method of claim 58, wherein the sample is obtained at about 15 weeks or about 10 weeks after the subject's last monthly period.

65. The method of claim 58, wherein a first sample is obtained between 4 and 25 weeks from the subject after the subject's last monthly period and a second sample is obtained between 15 and 41 weeks after the subject's last monthly period.

66. The method of claim 58, wherein the first sample is obtained between 4 and 20 weeks after the subject's last monthly period and a second sample is obtained between 21 and 41 weeks after the subject's last monthly period.

67. The method of claim 58, wherein the control is the level of AMH in a woman with a normal obstetric outcome.

68. The method of claim 58, wherein the adverse pregnancy outcome is selected from the group consisting of preeclampsia, intrauterine growth restriction, preterm labor, premature rupture of the membrane, diabetes, multiple gestation, and preterm delivery.

69. The method of claim 58, wherein the subject is a human subject or a non-human subject.

70. A method of evaluating the risk of a preterm delivery in a subject, comprising:
determining the level of AMH in a sample from a pregnant subject,
wherein if the subject has an abnormal level of AMH as compared to a predetermined normal level, the subject is more likely to have a preterm delivery, and if the subject does not have an abnormal level of AMH, the subject is less likely to have a preterm delivery.

71. The method of claim 70, wherein the sample is a blood sample.

72. The method of claim 70, wherein the level of AMH in the sample is determined immunologically.

73. The method of claim 72, wherein the level of AMH in the sample is determined by ELISA.

74. A method of predicting an adverse pregnancy outcome in a subject, comprising:
determining the level of AMH in a blood sample from a pregnant subject;
comparing mean AMH level of the sample to a predetermined normal level,
wherein an abnormal level of AMH in the sample compared to the predetermined normal level is predictive of an increased risk for an adverse pregnancy outcome compared to control, and
wherein the adverse pregnancy outcome comprises preeclampsia, intrauterine growth restriction, preterm labor, premature rupture of the membrane, diabetes, multiple gestation, or preterm delivery.

75. The method of claim 74, wherein the abnormal level is higher than the predetermined normal level.

76. The method of claim 74, wherein the abnormal level is lower than the predetermined normal level.

77. The method of claim 74, wherein the level of AMH is determined immunologically.
